SEQUENCE LISTING (1 sequence)

SEQ ID NO: 1 - protein, 429 aa, Artificial Sequence
  Foxp3
MPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

(19) United States
(12) Patent Application Publication
(10) Pub. No.: US 2020/0181273 A1
(43) Pub. Date: Jun. 11, 2020

(54) USE OF DENDRITIC CELLS EXPRESSING FOXP3 FOR DIAGNOSIS OR TREATMENT OF CANCER

(71) Applicant: RESEARCH & BUSINESS FOUNDATION SUNGKYUNKWAN UNIVERSITY, Suwon-si (KR)

(72) Inventors: Yong Soo BAE, Suwon-si (KR); Yi Deul JEONG, Suwon-si (KR); Myong Ho KANG, Suwon-si (KR)

(21) Appl. No.: 16/608,817

(22) PCT Filed: Apr. 30, 2018
(86) PCT No.: PCT/KR2018/005026

§ 371 Date: Oct. 26, 2019

Related U.S. Application Data
(60) Provisional application No. 62/491,320, filed on Apr. 28, 2017.

(51) Int. Cl. C07K 16/28; A61K 47/64; A61P 35/00

(52) U.S. Cl. CPC C07K 16/2866; A61K 38/00; A61P 35/00; A61K 47/642

(57) ABSTRACT

Provided is a use of at least one selected from the group consisting of Forkhead box P3 (Foxp3)-expressing dendritic cells and cluster of differentiation 8 (CD8)-positive regulatory T cells as a target for cancer therapy and/or as a marker for cancer diagnosis.

### TECHNICAL FIELD

Provided is a use of at least one selected from the group consisting of Forkhead box P3 (Foxp3)-expressing dendritic cells and cluster of differentiation 8 (CD8)-positive regulatory T cells as a target for cancer therapy and/or as a marker for cancer diagnosis.

### BACKGROUND ART

Dendritic cells (DCs) are antigen-presenting cells (APCs) of the mammalian immune system, which act as important messengers between the innate and the adaptive immune systems.

Forkhead box P3 (Foxp3) is a transcriptional regulatory factor known to be involved in the development and function of regulatory T cells (Treg) (Hori, S., Nomura, T. & Sakaguchi, S. Control of regulatory T cell development by the transcription factor Foxp3. Science 299, 1057-1061, doi:10.1126/science.1079490 (2003)).

However, little is known about immune cells other than T cells, such as dendritic cells, in relation to Foxp3 expression and the medical utility thereof.

### DETAILED DESCRIPTION OF THE INVENTION

### Technical Problem

The disclosure identifies Foxp3-expressing dendritic cells in vivo in cancer patients (e.g., blood, tumor tissues, etc.) and provides a use thereof in the diagnosis and/or therapy thereof and/or in monitoring the prognosis of cancer therapy.

An aspect provides a use of Foxp3-expressing dendritic cells as a cancer therapy target and/or a cancer diagnosis marker.

Another aspect provides a pharmaceutical composition comprising an inhibitor against Foxp3-expressing dendritic cells as an effective ingredient for treatment of cancer. The pharmaceutical composition for treatment of cancer may be administered to cancer patients in which Foxp3-expressing dendritic cells are detected.

Another aspect provides a use of an inhibitor against Foxp3-expressing dendritic cells in cancer therapy. The use in cancer therapy may account for the application of the inhibitor to cancer patients in the tumor tissue or blood of which Foxp3-expressing dendritic cells are detected.

Another aspect provides a method for treatment of cancer, the method comprising a step of administering an inhibitor against Foxp3-expressing dendritic cells in a pharmaceutically effective amount to a cancer patient. The cancer patient may be a patient having Foxp3-expressing dendritic cells detected in the tumor tissue or blood thereof.

Another aspect provides a pharmaceutical composition comprising an inhibitor against Foxp3-expressing dendritic cells as an effective ingredient for inhibition of CD8-expressing regulatory T cell(s) (CD8 positive regulatory T cell(s); CD8⁺ Treg). Another aspect provides a use of an inhibitor against Foxp3-expressing dendritic cells in inhibiting CD8⁺ Treg. Another aspect provides a method for inhibiting CD8⁺ Treg, the method comprising a step of administering an inhibitor against Foxp3-expressing dendritic cells to a patient in need of inhibiting CD8⁺ Treg. The patient may be a patient having Foxp3-expressing dendritic cells detected in the tumor tissue or blood thereof.

Another aspect provides a use of CD8⁺ Treg as a cancer therapy target.

Another aspect provides a pharmaceutical composition comprising an inhibitor against CD8⁺ Treg as an effective ingredient for treatment of cancer. The pharmaceutical composition for treatment of cancer may be administered to a cancer patient in the tumor tissue or blood of which CD8⁺ Treg are detected.

Another aspect provides a use of an inhibitor against CD8⁺ Treg in cancer therapy. The use in cancer therapy may account for application of the inhibitor to a cancer patient having CD8⁺ Treg in the tumor tissue or blood thereof.

Another aspect provides a method for treatment of cancer, the method comprising a step of administering an inhibitor against CD8⁺ Treg to a cancer patient. The cancer patient may be a patient having CD8⁺ Treg detected in the tumor tissue or blood thereof.

Another aspect provides a method for screening an anticancer agent, the method comprising the steps of: contacting a candidate compound with Foxp3-expressing dendritic cells, CD8⁺ Treg, or both; and defining the candidate compound as a candidate for an anticancer agent in a case where the level of Foxp3-expressing dendritic cells and/or CD8⁺ Treg decreases.

Another aspect provides a composition for cancer diagnosis or cancer prognosis identification, the composition comprising an agent capable of detecting Foxp3-expressing dendritic cells. Another aspect provides a method for cancer diagnosis or cancer prognosis identification or for providing information for cancer diagnosis or cancer prognosis identification, the method comprising a step of detecting Foxp3-expressing dendritic cells in a biological sample isolated from a patient. The method for cancer diagnosis may further comprise a step of defining the patient as a cancer patient in a case where Foxp3-expressing dendritic cells are detected (present) or a step of identifying the progress of cancer, depending on changes in the level of Foxp3-expressing dendritic cells, after the detecting step.

Another aspect provides a method for preparing CD8⁺ Treg, the method comprising a step of co-culturing Foxp3-expressing dendritic cells and CD8-positive T cells (CD8⁺ T cells).

Another aspect provides a use of CD8⁺ Treg in immunosuppression and/or in preventing and/or treating autoimmune disease or transplant rejection, wherein the CD8⁺ Treg is prepared by co-culturing Foxp3-expressing dendritic cells and CD8⁺ T cells. The CD8⁺ Treg may be prepared according to the above-mentioned method for preparation of CD8⁺ Treg. Another aspect provides an immunosuppressant or a composition comprising the CD8⁺ Treg, prepared by the preparation method, as an effective ingredient for prevention and/or treatment of autoimmune disease or transplant rejection. Another aspect provides an immunosuppression method comprising a step of administering the CD8⁺ Treg, prepared by the preparation method, to a subject in need thereof or a method for preventing and/or treating autoimmune disease or transplant rejection, the method comprising a step of administering CD8⁺ Treg, prepared by the preparation method, to a subject in need thereof.

### Technical Solution

Based on the finding that tumors and tumorous environments induce Foxp3-expressing dendritic cells which, in turn, induce CD8⁺ Treg in the tumor and the cells thus induced suppress the activity of CTLs that rush to eliminate the tumor, resulting in the sustained growth of tumors whereas the removal of Foxp3-expressing dendritic cells decreases the expression of CTLA4, which represses CTL activity, leading to the induction of effective anticancer immunity and the remarkable inhibition of tumor growth thanks to the non-repressed tumor-specific CTL activity, the disclosure proposes a use of Foxp3-expressing dendritic cells in the diagnosis and/or treatment of cancer and a technology for cancer treatment by removing Foxp3-expressing dendritic cells.

Accordingly, an aspect provides a use of Foxp3-expressing dendritic cells as a cancer therapy target and/or a cancer diagnosis marker.

Another aspect provides a pharmaceutical composition comprising an inhibitor against Foxp3-expressing dendritic cells as an effective ingredient for treatment of cancer. The Foxp3-expressing dendritic cells may be present in the tumor tissue or blood of a cancer patient. The pharmaceutical composition for treatment of cancer may be configured to be administered to cancer patients in which Foxp3-expressing dendritic cells are detected.

Another aspect provides a use of an inhibitor against Foxp3-expressing dendritic cells in cancer therapy. The use in cancer therapy may account for the application of the inhibitor to cancer patients in the tumor tissue or blood of which Foxp3-expressing dendritic cells are detected.

Another aspect provides a method for treatment of cancer, the method comprising a step of administering an inhibitor against Foxp3-expressing dendritic cells in a pharmaceutically effective amount to a cancer patient or a step of depleting Foxp3-expressing dendritic cells from a cancer patient (e.g., blood and/or tumor tissues of the patient). The cancer patient may be a patient having Foxp3-expressing dendritic cells detected in the tumor tissue or blood thereof.

Another aspect provides a pharmaceutical composition comprising an inhibitor against Foxp3-expressing dendritic cells as an effective ingredient for inhibition of CD8⁺ Treg. Another aspect provides a use of an inhibitor against Foxp3-expressing dendritic cells in inhibiting CD8⁺ Treg. Another aspect provides a method for inhibiting CD8⁺ Treg, the method comprising a step of administering an inhibitor against Foxp3-expressing dendritic cells to a patient in need of inhibiting CD8⁺ Treg or a step of depleting Foxp3-expressing dendritic cells from the patient (e.g., the blood and/or tumor tissue of the patient). The CD8⁺ Treg may be derived in the blood of a cancer patient by Foxp3-expressing dendritic cells. The patient may be a patient having Foxp3-expressing dendritic cells detected in the tumor tissue or blood thereof or having CD8⁺ Treg derived in the tumor tissue or blood therefor by Foxp3-expressing dendritic cells.

Another aspect provides a use of CD8⁺ Treg as a cancer therapy target.

Another aspect provides a pharmaceutical composition comprising an inhibitor against CD8⁺ Treg as an effective ingredient for treatment of cancer. The pharmaceutical composition for treatment of cancer may be administered to a cancer patient in the tumor tissue or blood of which CD8⁺ Treg are detected.

Another aspect provides a use of an inhibitor against CD8⁺ Treg in cancer therapy. The use in cancer therapy may account for application of the inhibitor to a cancer patient having CD8⁺ Treg in the tumor tissue or blood thereof.

Another aspect provides a method for treatment of cancer, the method comprising a step of administering an inhibitor against CD8⁺ Treg to a cancer patient or a step of depleting CD8⁺ Treg from the patient (e.g., blood and/or cancer tissue of the patient). The cancer patient may be a patient having CD8⁺ Treg detected in the tumor tissue or blood thereof.

Another aspect provides a method for screening an anticancer agent, the method comprising the steps of: contacting a candidate compound with Foxp3-expressing dendritic cells, CD8⁺ Treg, or both; and defining the candidate compound as a candidate for an anticancer agent in a case where the level of Foxp3-expressing dendritic cells and/or CD8⁺ Treg decreases. In detail, the anticancer agent-screening method may comprise the steps of: (1) contacting a candidate compound with Foxp3-expressing dendritic cells, CD8⁺ Treg, or both, or a biological sample containing the same (e.g., blood, corpuscles, tumor tissue, etc.); and (2) measuring levels of Foxp3-expressing dendritic cells and/or CD8⁺ Treg. The anticancer agent-screening method may comprise, after step (2), a step of comparing the levels of Foxp3-expressing dendritic cells and/or CD8⁺ Treg between measurements in step 2 and before treatment with the candidate compound (step (3). In addition, the anticancer agent-screening method may comprise, after step (2) or (3), a step of defining the candidate compound as an anticancer agent candidate in a case where the levels of Foxp3-expressing dendritic cells and/or CD8⁺ Treg in step (2) are lower than those measured before treatment with the candidate compound (step (4)). The steps of the screening method may be each performed in vitro. In addition, Foxp3-expressing dendritic cells and/or CD8⁺ Treg may be cells isolated from a living organism.

Another aspect provides a cancer diagnosis composition comprising an agent capable of detecting Foxp3-expressing dendritic cells. Another aspect provides a method for cancer diagnosis or cancer prognosis identification or for providing information for cancer diagnosis or cancer prognosis identification, the method comprising a step of detecting Foxp3-expressing dendritic cells in a biological sample isolated from a patient. The method for cancer diagnosis or cancer prognosis identification may further comprise a step of defining the patient as a cancer patient in a case where Foxp3-expressing dendritic cells are detected (present) or a step of identifying the progress of cancer, depending on changes in the level of Foxp3-expressing dendritic cells, after the detecting step. In the cancer diagnosis method, the biological sample may include blood, corpuscles, and the like isolated from a mammalian animal, such as a human, in need of identifying prognosis after the onset of cancer. According to an embodiment, the cancer diagnosis method may further comprise a step of administering a pharmaceutically effective amount of at least one selected from the group consisting of an inhibitor against Foxp3-expressing dendritic cells and an inhibitor against CD8⁺ Treg to the defined cancer patient, after the step of defining the patient as a cancer patient.

In the method for cancer prognosis identification, the biological sample may be at least one selected from the group consisting of blood, corpuscles, and tumor tissues which are all isolated from a cancer patient to be identified (monitored) for cancer prognosis (progress). In the method for cancer prognosis identification, when levels of Foxp3-expressing dendritic cells in the biological sample isolated from a cancer patient have been measured at two or more different times, the cancer patient is identified to be under cancer aggravation or accelerated cancer progression in a case where the level of Foxp3-expressing dendritic cells measured at a temporal point is higher than that measured at an earlier time while the cancer patient is identified to be under cancer alleviation or delayed cancer progression in a case where the level of Foxp3-expressing dendritic cells measured at a temporal point is lower than that measured at an earlier time. The method for cancer prognosis identification may comprise the steps of: (1) measuring levels of Foxp3-expressing dendritic cells in a biological sample isolated from a cancer patient at two or more different times; and (2) determining cancer aggravation or accelerated cancer progression in a case where the level of Foxp3-expressing dendritic cells, measured at a temporal point, is higher than that measured at an earlier time and cancer alleviation or delayed cancer progression in a case where the level of Foxp3-expressing dendritic cells is lower than that measured at an earlier time.

The method for cancer prognosis identification may be applied to monitoring the efficacy of anticancer therapy (monitoring post-treatment prognosis) in a patient who is under anticancer therapy (e.g., administered an anticancer agent). Thus, contemplated according to another aspect of the present disclosure is a composition for identifying (monitoring) efficacy of anticancer therapy, which comprises an agent capable of detecting Foxp3-expressing dendritic cells. Another aspect provides a method for identifying (monitoring) anticancer therapy efficacy or for providing information on the identification (monitoring) of anticancer therapy efficacy, the method comprising a step of detecting Foxp3-expressing dendritic cells in a biological sample isolated from a patient. In the method for identifying (monitoring) anticancer therapy efficacy, the patient may be a patient to whom anticancer therapy has been applied, the anticancer therapy may be a single therapy or a combined therapy of two or more selected from the group consisting of chemotherapy such as administration of an anticancer agent, biological therapy such as gene therapy, physical therapy such as radiotherapy, and surgical operation, and the biological sample may be at least one selected from blood, corpuscles, and a tumor tissue, which are all isolated from a cancer patient who is to be monitored for anticancer therapy efficacy. In the method for identifying anticancer therapy efficacy, the anticancer therapy is identified to have no anticancer effects in a case where the level of Foxp3-expressing dendritic cells in the biological sample isolated from a patient who has been under the anticancer therapy is increased, compared to that measured before the anticancer therapy while being identified to have an advantageous anticancer effect in a case where the level of Foxp3-expressing dendritic cells in the biological sample isolated from a patient who has been under the anticancer therapy is decreased, compared to that measured before the anticancer therapy. The method for identifying anticancer therapy efficacy may comprise the steps of: (1) measuring levels of Foxp3-expressing dendritic cells in a biological sample isolated from a cancer patient before and after the application of cancer therapy to the cancer patient; and (2) identifying the anticancer therapy to be ineffective for the cancer patient in a case where the level of Foxp3-expressing dendritic cells, measured after the anticancer therapy, is higher than that measured before the anticancer therapy or to be effective for the cancer patient in a case wherein the level of Foxp3-expressing dendritic cells, measured after the anticancer therapy, is lower than that measured before the anticancer therapy. As concerns the time at which levels of Foxp3-expressing dendritic cells are measured, “after anticancer therapy” may account for any one duration within two months following anticancer therapy (for example, eight weeks following anticancer therapy, seven weeks following anticancer therapy, six weeks following anticancer therapy, five weeks following anticancer therapy, four weeks following anticancer therapy, three weeks following anticancer therapy, two weeks following anticancer therapy, or one week following anticancer therapy). The method for identifying anticancer therapy efficacy may comprise, after step (3), a step of (4) ceasing the anticancer therapy in the cancer patient or applying a different kind of anticancer therapy to the cancer patient in a case wherein the level of Foxp3-expressing dendritic cells, measured after the anticancer therapy, is higher than that measured before the anticancer therapy (in a case where the anticancer therapy is identified to be ineffective for the cancer patient) or maintaining or enhancing the anticancer therapy in a case wherein in a case wherein the level of Foxp3-expressing dendritic cells, measured after the anticancer therapy, is lower than that measured before the anticancer therapy (in a case where the anticancer therapy is identified to be effective for the cancer patient). As used herein, the term “anticancer therapy efficacy” may be intended to encompass all events of removing or alleviating (turning around) symptoms of cancer, such as apoptosis or growth inhibition of cancer cells, extinction or size reduction of cancer tissues, inhibition of cancer metastasis, etc.

Another aspect provides a method for preparing CD8⁺ Treg, the method comprising a step of co-culturing Foxp3-expressing dendritic cells and CD8⁺ T cells. The co-culturing step may be carried out by co-culturing Foxp3-expressing dendritic cells and CD8⁺ T cells at a cell population ratio of 1:0.1-10, 1:0.1-8, 1:0.1-6, 1:0.1-4, 1:0.1-2, 1:0.1-1, 1:0.3-10, 1:0.3-8, 1:0.3-6, 1:0.3-4, 1:0.3-2, 1:0.3-1, 1:0.5-10, 1:0.5-8, 1:0.5-6, 1:0.5-4, 1:0.5-2, 1:0.5-1, 1:0.8-10, 1:0.8-8, 1:0.8-6, 1:0.8-4, 1:0.8-2, 1:0.8-1, 1:1-10, 1:1-8, 1:1-6, 1:1-4, or 1:1-2 (Foxp3-expressing dendritic cells : CD8⁺ T cells).

Another aspect provides CD8⁺ Treg prepared by co-culturing Foxp3-expressing dendritic cells and CD8⁺ T cells. The CD8⁺ Treg may be the cells prepared according to the above-mentioned method for preparing CD8-expressing regulatory T cells.

Another aspect provides a use of CD8⁺ Treg in immunosuppression and/or in preventing and/or treating autoimmune disease or transplant rejection, wherein the CD8⁺ Treg are prepared by co-culturing Foxp3-expressing dendritic cells and CD8⁺ T cells. The CD8⁺ Treg may be prepared according to the above-mentioned method for preparation of CD8⁺ Treg. Another aspect provides an immunosuppressant or composition comprising the CD8⁺ Treg, prepared by the preparation method, as an effective ingredient for prevention and/or treatment of autoimmune disease or transplant rejection. Another aspect provides an immunosuppression method comprising a step of administering the CD8⁺ Treg, prepared by the preparation method, to a subject in need thereof, or a method for preventing and/or treating autoimmune disease or transplant rejection, the method comprising a step of administering CD8⁺ Treg, prepared by the preparation method, to a subject in need thereof. The autoimmune disease may be selected from rheumatism, lupus, autoimmune hepatitis, and autoimmune hemolytic anemia.

Hereinafter, a detailed description will be given of the disclosure.

Foxp3 (Forkhead box P3), also known as scurfin, is a protein involved in immune system responses. Foxp3 functions as a master regulator of the regulatory pathway in the development and function of regulatory T cells. The Foxp3 may be derived from mammals including primates such as humans, apes, etc. and rodents such as rats, mice, etc. Examples may include human Foxp3 (e.g., GenBank Accession No. NP_001107849.1 (gene (mRNA): NM_001114377.1), NP_054728.2 (gene (mRNA): NM_014009.3)), and mouse Foxp3 (e.g., GenBank Accession No. NP_001186276.1 (gene (mRNA): NM_001199347.1), NP_001186277.1 (gene (mRNA): NM_001199348.1), NP_473380.1 (gene (mRNA): NM_054039.2)). In an embodiment, the Foxp3 may comprise the amino acid sequence of SEQ ID NO: 1 (MPNPRPAKPMAPSLALGPSPGVLPSWKTAPKGSELLGTRGSGGPFQGRDLRSG AHTSSSLNPLPPSQLQLPTVPLVMVAPSGARLGPSPHLQALLQDRPHFMHQLSTV DAHAQTPVLQVRPLDNPAMISLPPPSAATGVFSLKARPGLPPGINVASLEWVSRE PALLCTFPRSGTPRKDSNLLAAPQGSYPLLANGVCKWPGCEKVFEEPEEFLKHC QADHLLDEKGKAQCLLQREVVQSLEQQLELEKEKLGAMQAHLAGKMALAKAP SVASMDKSSCCIVATSTQGSVLPAWSAPREAPDGGLFAVRRHLWGSHGNSSFPE FFHNMDYFKYHNMRPPFTYATLIRWAILEAPERQRTLNEIYHWFTRMFAYFRNH PATWKNAIRHNLSLHKCFVRVESEKGAVWTVDEFEFRKKRSQRPNKCSNPCP), but is not limited thereto.

Dendritic cells (DCs) are immune cells of the mammalian immune system, functioning as antigen-presenting cells. In the disclosure, DCs may be derived from mammals including primates such as humans, apes, etc. and rodents such as rats, mice, etc. In an embodiment, DCs may be derived (isolated) from blood (corpuscles) of mammals, for example, humans (e.g., cancer patients).

An inhibitor against Foxp3-expressing dendritic cells may be any agent that can reduce a level of Foxp3-expressing dendritic cells, or kill or remove Foxp3-expressing dendritic cells in a subject to be administered (in vivo (e.g., blood and/or tumor tissues of cancer patients), biological samples isolated from patients (e.g., isolated blood and/or tumor tissues)). For example, the inhibitor may be at least one selected from the group consisting of antibodies specific for Foxp3-expressing dendritic cells, cytotoxic drugs, antibody-cytotoxic drug conjugates, antibody-magnetic particle composites and the like, or may be in form of a nano-delivery system comprising the at least one inhibitor, but is not limited thereto. The term “nano-delivery system”, as used herein, refers to a nano-size particle (e.g., 1-1000 nm) encapsulating or delivering the inhibitor. It may be made of at least one material selected from the group consisting of proteins, lipids, and other biocompatible or biodegradable polymers, without morphological limitations thereto. Cluster of differentiation 8 (CD8) is a transmembrane glycoprotein that serves as a co-receptor for the T cell receptor (TCR). Like the TCR, CD8 binds to a major histocompatibility complex (MHC), but is specific for the class I MHC protein. CD8 may be derived from mammals including primates such as humans, apes, etc. and rodents such as rats, mice, etc. For example, the CD8 may be human CD8 (e.g., GenBank Accession No. NP_001139345.1 (gene (mRNA): NM_001145873.1), NP_001759.3 (gene (mRNA): NM_001768.6), NP_741969.1 (gene (mRNA): NM_171827.3), NP_001171571.1 (gene (mRNA): NM_001178100.1), NP_004922.1 (gene (mRNA): NM_004931.4), NP_742099.1 (gene (mRNA): NM_172101.3), NP_742100.1 (gene (mRNA): NM_172102.3), NP_757362.1 (gene (mRNA): NM_172213.3) etc.).

T cells are a type of lymphocytes that accounts for antigen-specific adaptive immunity. Regulatory T cells (Treg) are a subpopulation of T cells that maintain tolerance to self-antigens and prevent autoimmune disease. In the present disclosure, CD8⁺ T cells and CD8⁺ regulatory T cells (CD8⁺ Treg) may be derived from mammals including primates such as humans, apes, etc. and rodents such as rats, mice, etc. In an embodiment, the T cells may be derived (isolated) from mammals, e.g., blood of humans (e.g., cancer patients).

An inhibitor against CD8⁺ Treg may be any agent that can reduce a level of CD8⁺ Treg or remove CD8⁺ Treg in a subject to be administered (in vivo (e.g., blood and/or tumor tissues of cancer patients), biological samples isolated from patients (e.g., isolated blood and/or tumor tissues)). For example, the inhibitor may be at least one selected from the group consisting of antibodies specific for CD8⁺ Treg, cytotoxic drugs, antibody-cytotoxic drug conjugates, antibody-magnetic particle composites, and the like, or may be in form of a nano-delivery system comprising the at least one inhibitor, but is not limited thereto. The term “nano-delivery system” refers to a nano-size particle (e.g., 1-1000 nm) encapsulating or delivering the inhibitor. It may be made of at least one material selected from the group consisting of proteins, lipids, and other biocompatible or biodegradable polymers, without morphological limitations thereto.

As used herein, the “patient” may be a mammal including a primate such as a human, an ape, etc. and a rodent such as a mouse, a rat, etc. or may be cells or tissues (e.g., blood, corpuscles, tumor tissues, etc.) isolated from the mammal. In one embodiment, the patient may be a cancer patient or cells or tissues (e.g., blood, corpuscles, tumor tissues, etc.) isolated from the cancer patient. For example, the patient may be a cancer patient in which Foxp3-expressing dendritic cells, CD8⁺ Treg, or both are detected.

In addition, a biological sample used for cancer diagnosis may be cells, a tissue, or body fluid (e.g., blood, corpuscles, tumor tissues, etc.) isolated from mammals (including primates such as humans, apes, etc. and rodents such as mice, rats, etc.).

The cancer that the treatment and/or diagnosis of the present disclosure may be applied to may be any solid cancer or blood cancer. By way of example, the cancer may be at least one selected from the group consisting of squamous cell carcinoma, lung cancer (e.g., small-cell lung cancer, non-small-cell lung cancer, adrenocarcinoma of lung, squamous cell carcinoma of lung, etc.), peritoneal cancer, skin cancer, rectal cancer, perianal cancer, esophagus cancer, small intestine cancer, endocrine gland cancer, parathyroid cancer, adrenal cancer, soft-tissue sarcoma, urethral cancer, chronic or acute leukemia, lymphocytic lymphoma, hepatoma, gastric cancer, pancreatic cancer, cervical cancer, ovarian cancer, bladder cancer, breast cancer, colon cancer, colorectal carcinoma, endometrial carcinoma, uterine carcinoma, salivary gland tumor, prostate cancer, vulvar cancer, thyroid cancer, head or neck cancer, brain cancer, and osteosarcoma, but is not limited thereto. In an embodiment, the cancer may be a solid cancer such as colorectal cancer, gastric cancer, lung cancer, pancreatic cancer, breast cancer, etc. and/or a blood cancer such as lymphoma, leukemia, etc. The cancer may include a metastatic cancer as well as a primary cancer.

In the present disclosure, the term “cancer therapy” or “treatment of cancer” is intended to encompass all actions that elicit the effect of suppressing the growth of cancer cells or killing (eliminating) cancer cells as well as the effect of preventing the aggravation of cancer by inhibiting the migration, invasion, and metastasis of cancer cells.

The agent capable of detecting Foxp3-expressing dendritic cells may be selected from all compounds (e.g., small-molecule chemicals, antibodies, etc.) binding specifically to Foxp3-expressing dendritic cells. For example, the agent may be a combination of at least one selected from small-molecule chemicals and antibodies, which bind specifically to Foxp3 expressed in dendritic cells and at least one selected from small-molecule chemicals and antibodies, which bind specifically to surface proteins of Foxp3-expressing dendritic cells, and a nano-delivery system including them (antibodies and/or small-molecule chemicals).

The agent capable of detecting CD8⁺ Treg may be selected from all compounds (e.g., small-molecule chemicals, antibodies, nano-delivery systems, etc.) that bind specifically to CD8⁺ Treg. For example, the agent may be at least one selected from small-molecule chemicals and antibodies, which bind specifically to surface proteins of CD8⁺ Treg.

The agent capable of detecting Foxp3-expressing dendritic cells and/or the agent capable of detecting CD8⁺ Treg may be labeled with a typical marker that can be detected by a typical method (e.g., enzymatic reaction, fluorescence, luminescence and/or radiation). For example, the marker may be at least one selected from the group consisting of fluorescents (e.g., fluorescent dye, fluorescent proteins, etc.), luminescent materials, and radioisotopes, but is not limited thereto. In one embodiment, the detection of Foxp3-expressing dendritic cells and/or CD8⁺ Treg may be carried out using flow cytometry, fluorescence-activated cell sorting (FACS), immunochromatography, immunohistochemical staining, enzyme-linked immunosorbent assay (ELISA), radioimmunoassay (RIA), enzyme immunoassay (EIA), fluorescence immunoassay (FIA), luminescence immunoassay (LIA), or Western blotting, without limitations thereto.

In the method for screening an anticancer agent, the candidate compound may be selected from the group consisting of various compounds, for example, small-molecular chemicals, proteins, polypeptides, oligopeptides, polynucleotides, oligonucleotides, and plant or animal extracts.

### Advantageous Effects

Provided is a use as a cancer diagnosis marker and/or a cancer therapy target of Foxp3-expressing dendritic cells within a tumor or tumor environments (e.g., blood of cancer patients) and/or CD8⁺ Treg derived thereby. The cells can find applications in a broad spectrum of fields including the diagnosis and treatment of cancer, research into anticancer agents, the prognosis monitoring after anticancer therapy, and the like.

### BRIEF DESCRIPTION OF THE DRAWINGS

FIG. 1 is a plot of proportions of Foxp3-expressing dendritic cells (fxDC) in blood (% of fxDC/CD11c⁺DC) of tumor mouse models under tumor growth (Paired one-way ANOVA without multiple comparison correction).

FIG. 2a is a graph showing fxDC distributions in blood (% of fxDC/b-DC) of normal persons and cancer patients (n=30 human samples, unpaired one-way ANOVA without multiple-comparisons correction).

FIG. 2b is a graph showing fxDC distributions in blood in various tumor mouse models (EL4; lymphoma, B16; melanoma, LLC; Lewis lung carcinoma, 266-6; pancreatic cancer, CT-26; colon cancer, 4T-1; breast cancer, RENCA; renal cancer; n=5 to 7 mice per tumor model, unpaired one-way ANOVA without multiple-comparisons correction).

FIG. 3 shows fxDC distributions in blood of dendritic cell-specific Foxp3-knockout mice (CD11c-Cre×Foxp3^{fl/fl}: hereinafter referred to as Foxp3^{cKO} mice) and floxed littermates (Foxp3^{fl/fl}).

FIG. 4 shows plots of tumor volumes vs. times (left) and tumor weights after tumor transplantation (right) in wild-type (WT) mice (Foxp3^{fl/fl}) and Foxp3^{cKO} mice (n=5 mice each, unpaired one-tailed t-test.)

FIG. 5 shows measurements of fxDC in tumor tissues of WT mice and Foxp3^{cKO} mice (E=5, unpaired one-tailed t-test. ***p<0.001).

FIG. 6 is a plot of tumor volumes against time in WT mice and Foxp3^{cKO} mice with various solid cancers.

FIG. 7 shows proportions of cytotoxic CD8⁺ T-cells in tumor tissues of Foxp3^{cKO} mice (n=3, unpaired one-tailed t-test).

FIG. 8 shows the cytotoxicity of CD8⁺ T-cells against tumor cells in tumor tissues of Foxp^{cKO} mice as measured for activity of CTL (Cytotoxic T Lymphocytes) (n=3, unpaired one-tailed t-test).

FIG. 9 shows expression levels of CTLA4 (cytotoxic T-lymphocyte-associated protein 4) in CD8⁺ T cells in tumor tissues of WT mice and Foxp3^{cKO} mice.

FIG. 10 shows proportions of CTLA4-expressing CD8⁺ T cells (CTLA4⁺ CD8⁺ T cells) in CD8⁺ T cells in tumor tissues of WT mice and Foxp3^{cKO} mice (unpaired one-tailed t-test, **p<0.01 and ***p<0.001).

FIG. 11 is a plot of tumor cell (EL4)-targeting CTL activities of CTLA4⁺ CD8⁺ T cells and CTLA4⁻ CD8⁺ T cells isolated from EL4 tumor (unpaired one-tailed t-test).

FIG. 12 shows Foxp3⁺ CD8⁺ Treg distributions after co-culture of fxDC and CD8⁺ T cells (unpaired one-tailed t-test).

FIG. 13 shows potentials of fxDC and Foxp3-depleted DC to induce CD4/8 Treg, wherein pre-activated T cells of Foxp3^{GFP} mice were co-cultured with splenic DCs (spDC), blood DCs (bDC) and fxDC-depleted (DT-treated) bDCs (bDC/DT) of TB Foxp3^{DTR} mice (p3/E, for b-DCs), and the population of Foxp3⁺ CD4⁺ and CD8⁺ T cells was examined E=3, unpaired two-way ANOVA with multiple comparisons.

FIG. 14 is a plot showing proportions of fxDC and CD8⁺ Tregs cells in blood of TB mice (n=27).

FIG. 15 shows CD4⁺ /CD8⁺ Treg distributions in tumor tissues of WT mice and Foxp3^{cKO} mice (unpaired two-way ANOVA with multiple comparisons).

FIG. 16 shows T cell growth levels after co-culture of T cells and CD8⁺ /CD4⁺ Treg cells.

FIG. 17 shows IFN-gamma⁺ T cell levels after co-culture of T cells and CD8⁺/CD4⁺ Treg cells (unpaired one-way ANOVA with multiple-comparisons correction. *p<0.05, **p<0.01).

FIG. 18 shows CTLA4-expressing T cell levels after co-culture of CD8⁺ Treg and CD8⁺ T cells (unpaired one-tailed t-test, *p<0.05, **p<0.01, ***p<0.001).

FIG. 19 shows CTLA4⁺ CD8⁺ T cell levels after co-culture of WT CD8⁺ T cells and DT-treated to-CD8⁺ T-cells or PBS-treated to-CD8⁺ T-cells (n=3, unpaired one-tailed t-test).

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### Reference Example

1. Preparation of Mice

Mice 7-10 weeks old, including wild-type mice C57BL6 and BALB/c and genetically modified mice C57BL6-OT-1, C57BL6-Foxp3^{GFP} (Foxp3-GFP reporter mice expressing Foxp3-green fluorescent protein (GFP) fusion protein in Foxp3⁺ cells), C57BL6-Foxp3^{DTR} (Foxp3-DTR transgenic (Tg) mice expressing a diphtheria toxin receptor (DTR), instead of a Foxp3-encoding exon, under the control of a Foxp3 promoter), C57BL6-Foxp3^{DTR-GFP} (prepared by backcrossing C57BL6-Foxp3^{DTR} with C57BL6-Foxp3^{GFP} for three generations), C57BL6-Foxp3-floxed (Foxp3^{fl/fl}), CD11c-Foxp3^{cKO} (C57BL6-Foxp3^{cKO} prepared by crossing CD11c-cre with Foxp3^{fl/fl}), C57BL6-Rag1^{tmlMom} (RAG1^{-/-}), and CD11c-cre were used in the following experiments. C57BL6-OT-1, Foxp3^{GFP}, Foxp3^{DTR,} Rag1^{-/-}, and CD11c-cre were purchased from Jackson Laboratory (Bar Harbor, Sacramento, Calif.). Foxp3-floxed (C57BL6-Foxp3^{fl/fl}) was provided by A. Rudensky, Memorial Sloan Kettering Cancer Center, NY. All the mice were maintained and managed in the specific pathogen-free (SPF) animal care facility according to the Institute/University Animal Care and Use guidelines (Sungkyunkwan University). For the experiments, the mice were transferred to separate animal care chambers and co-housed in the same condition. The DTR mice were treated with diphtheria toxin (DT) as reported previously (refer to “Kim, J et al. Cutting edge: depletion of Foxp3+ cells leads to induction of autoimmunity by specific ablation of regulatory T cells in genetically targeted mice. J Immunol 183, 7631-7634, doi:10.4049/jimmunol.0804308 (2009)” and “Penaloza-MacMaster, P. et al. Interplay between regulatory T cells and PD-1 in modulating T cell exhaustion and viral control during chronic LCMV infection. The Journal of experimental medicine 211, 1905-1918, doi:10.1084/jem.20132577 (2014)”). In brief, a solution of DT in PBS was i.p. injected at a dose of 200 μl (50 μg/kg) into Foxp3^{DTR} or Foxp3^{DTR-GFP} mice for three consecutive days (day-3, day-2, and day-1) before blood sampling, after which CD11c⁺MHC⁺ dendritic cells (DCs) were isolated from the blood or tumors of the DT-treated mice and used in the following tests.

2. Preparation of Mouse Cell Lines and Human Primary Cells

EL4 (C57BL/6 mouse-derived lymphoma), EG7 (OVA-expressing EL4), B16/F10 (C57BL/6 mouse-derived skin melanoma), 266-6 (C57BL/6 mouse-derived pancreatic acinar cell tumor), CT26 (BALB/c mouse-derived colon carcinoma), 4T-1 (BALB/c mouse-derived mammary carcinoma), and RENCA (BALB/c mouse-derived renal adenocarcinoma) cells were purchased from the American Type Culture Collection (ATCC). According to the protocol approved by the Samsung Medical Center IRB (# SMC 2016-04-057), human peripheral blood mononuclear cells (hPBMCs) were taken from patients with malignant tumor (glioblastoma (GBM, stages 3 and 4), colon cancer (CC, stage 2(CC2), 3(CC3), and 4(CC4)) and gastric cancer (GC, stage 2(GC2), 3(GC3), and 4(GC4)), and healthy donors.

3. Construction of Mouse Tumor Model

Mouse tumor models were constructed by injecting EL4/EG7, B16/F10, LLC, 266-6, CT-26, 4T-1, and RENCA cells at a dose of 5×10⁵ cells into right flanks of wild-type (wt) mice (C57BL6 and BALB/c) and genetically modified mice (C57BL6-Foxp3^{GFP}, C57BL6-Foxp3^{DTR}, and C57BL6-Foxp3^{cKO} (Foxp3^{fl/fl}xCD11c-cre)).

3. Isolation of Primary Immune Cells

Mouse PBMCs and tumor infiltrated leukocytes (TILs) were isolated by Ficoll (GE Healthcare, Little Chalfont, UK) and Percoll (Sigma Aldrich, Chemie GmbH, Taufkirchen, Germany) density gradient centrifugation from blood and tumor tissues of TB mice. After depletion of lineage⁺ (CD3⁺/CD14⁺/CD19⁺) cells, dendritic cells were separated from TILs or PBMCs of Foxp3^{GFP} mice by using CD11c-microbeads (Miltenyi Biotech). Because of the small tumor sizes of Foxp3^{cKO} mice, TILs isolated from 5 to 10 TB Foxp3^{cKO} mice were pooled for a single test after normalization (expressed as p5/E or p10/E). Myeloid derived suppressor cell (MDSC) subsets were isolated from blood of TB Foxp3^{GFP} mice or control Foxp3^{GFP} mice with the aid of MDSC Isolation Kit (Miltenyi Biotec). From blood or tumor tissues of Foxp3^{GFP} mice, Foxp3⁺ fxDCs, cDCs, CD4⁺ Treg, CD8⁺Tregs, CTLA4⁺/CTLA4⁻ T-cells, and CCR2⁺ /CCR2⁻ cells were isolated using BD FACSAria™II. All in vitro and adoptive transfer (AT) tests were conducted after the normalization of isolated cells.

4. Flow Cytometry

For phenotype analysis, immunofluorescent staining was carried out. Cells were stained with proper antibodies at 4° C. for 20 min in FACS buffer. A purchase was made of FITC-labeled anti-mouse antibodies [Ly6g (1A8), CD11c (N418), I-A/I-E (M5/114.15.2), CD3 (17A2), and B220 (RA3-6B2)] were purchased from Thermo Fisher-eBioscience (Waltham, Mass., USA), an anti-mouse CD14 (Sa14-2) antibody from Biolegend (San Diego, Calif., USA), a phycoerythrin (PE)-labeled anti-mouse Foxp3 antibody (150D) from Biolegend), an anti-mouse zbtb46 antibody (U4-1374) from BD biosciences (San Jose, Calif., USA), and PE-labeled anti-mouse antibodies (Ly6c (HK1.4), CD11c (N418), CD317 (BST2, 927), ki-67 (SolA15), and CD25 (PC61.5) from Thermo Fisher-eBioscience. PerCP-Cy5.5-labeled anti-mouse antibodies [CD11b, Gr-1 (RB6-8C5), CD44 (IM7), Foxp3 (FJK-16s), I-A/I-E, CD11c, and CD25 (PC61.5)], PE-Cy7-labeled anti-mouse antibodies [CD4 (GK1.5), CD8a (53-6.7), F4/80 (BM8), CD16/CD32 (93), Foxp3 (FJK-16s), and CD11c (N418)], APC-labeled anti-mouse antibodies [CD3 (17A2), CD14 (SA14-2), CD19 (1D3/CD19), Foxp3 (FJK-16s), CCR2 (475301), CTLA4 (UC10-4B9) and CD44 (IM7)], and pacific blue-labeled anti-mouse antibodies [CD4 (GK1.5), CD8a (53-6.7), CD3 (17A2) and CD62L (MEL-14)] were purchased from Thermo Fisher-eBioscience. All samples were also stained with isotype control antibodies. After being washed, the cells were analyzed using FACSCanto II (BD Biosciences, San Jose, Calif., USA) and FACS DIVA software. Antibodies to Foxp3, IFN-gamma (XMG1.2), perforin, and Granzyme B were purchased from Thermo Fisher-eBioscience and used for intracellular staining according to the manufacturer's protocols.

5. FACS Gating Strategy for Foxp3-Expression Dendritic Cells (fxDCs)

Mouse PBMCs and TILs (tumor infiltrated leukocytes) were isolated from tumor and blood of TB mice. The isolated cells were stained with proper antibodies in cell staining buffer. Antibody panels were designed and constructed to be optimized for respective gating strategies depending on detection channels of flow cytometry. Compensations were performed with single-stained UltraComp eBeads (Affymetrix) or cells. For all channels, positive and negative cells were gated from Fluorescence Minus One controls (FMOs) and isotype controls. For Foxp3^{GFP} mice, Foxp3⁺ was gated using GFP littermate control. For wt TB mice, intracellular staining was performed in Foxp3⁺ cells. fxDC gating was performed as follows; FVD⁺ (live cells), CD45⁺, Lineage (CD3/CD19/CD14; T-cells, B-cells and Monocytes)-negative, CD11c⁺, MHC and Foxp3⁺. All phenotype panels of fxDCs were constructed the gating strategies as described above. FVD: Fixable Viability Dye.

6. Co-Culture of DC/T-Cells

T cells were separated and purified from spleens of TB Fopx3^{GFP} mice. In this regard, the spleens of TB Fopx3^{GFP} mice were homogenized in an RPMI medium and passed through a 70 μm nylon cell strainer (BD Falcon). Thereafter, an ACK lysis buffer (Lonza) was applied to the cell suspension to separate T cells. The separated T cells were purified using mouse CD4 and CD8 T-cell Isolation Kit II (Miltenyi Biotech) and then labeled at 37° C. with 5,6-carboxyfluorescein succinimidyl ester (CFSE, Molecular Probes) (at 1 mM for 10 min), Cell Trace Violet (CTV, Invitrogen) (at 10 μM for 15 min), or 4-chlorobenzenesulfonate salt (DiD, Thermo fisher) (at 5 μM for 15 min). CFSE/CTV-labeled T cells were incubated with anti-CD3/CD28 antibody (alpha-CD3 10 μg/ml, alpha-CD28 4 μg/ml) for one day, followed by co-culturing 5×10⁵ T cells together with fxDCs or other DC subsets at a ratio of 1:5 (DC:T) for three days. Cell proliferation was measured using flow cytometry (Reference Example 4). As for OT-1 T cells (ovalbumin-specific, CD8⁺ T cells) to be co-cultured, splenic OT-1 T cells were prepared from OT-1 mice and labeled as stated above. Without additional stimulation, CFSE-labeled 5×10⁵ naive OT-1 T-cells were isolated from Foxp3^{DTR} tumor mice and co-cultured together with DT-treated (fxDC-depleted) bDCs, or PBS-treated (fxDC-containing) bDC, or sp-DCs at a ratio of 1:5 (DC:T).

7. CTL (Cytotoxic T Lymphocytes) Assay

On day 21 after tumor transplantation, CD8⁺ T-cells isolated from the tumor tissues of Foxp3^{fl/fl} or Foxp3^{cKO} TB mice were co-cultured with CTV-labeled target cells (1×10⁵ EL4 cells) at different ratios for 24 hours. After PI staining, flow cytometry was performed with reference to Reference Example 4 to analyze CTL activity. On day 21, tu-DCs isolated from tumor of Foxp^{fl/fl} or Foxp3^{cKO} TB mice were co-cultured with splenic CD8⁺ T-cells at a ratio of 1:5 (DC:T) for three days to produce CTLs which were then measured for activity. For this, CTLA4⁺ or CTLA4⁻ CD8⁺ T-cells were isolated from tumors of TB Foxp3^{GFP} mice with the aid of FACSAria™II and assayed for CTL activity.

8. Adoptive Transfer (AT) Assay

M-MDSCs (1×10⁶ cells) isolated using MDSC Isolation Kit (Miltenyi Biotec, Bergisch Gladbach, Germany) from the spleen or blood of TB Foxp3^{GFP} mice were transferred to control (tumor-free mice) or TB mice via the tail vein (adoptive transfer; AT). Three days after AT, fxDC was analyzed in the AT recipients.

AT of CD8⁺ T cells was performed. To this end, the cells were isolated using CD8⁺ T-cell isolation kit (Miltenyi Biotec) from the spleen of tumor-free mice or OT-1 mice, or the blood or tumor tissues of TB Foxp3^{fl/fl} and Foxp3^{cKO} and the isolated cells were labeled with CTV (10 μM) or DiD (10 μM) at 37° C. for 15 min. The labeled cells (1'10⁶ cells) were transferred as described above (AT).

Statistical Analysis

Statistical analysis was done using GraphPad 5.0 software, with statistical significance set at P<0.05 (*P<0.05, **P<0.01, ***P<0.001). All experiment results were obtained from at least three independent experiments (3E) which were each carried out in triplicate. Statistical data are expressed as mean±s.e.m.

### EXAMPLE 1

### Measurement of Blood fxDC in Tumor Patients

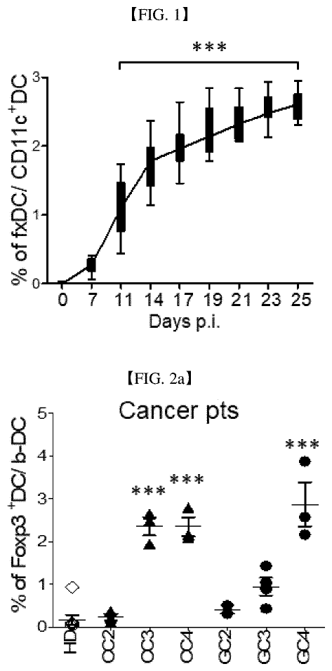

In mouse tumor models constructed by injecting EL4 lymphoma into mice (see Reference Example 2), orbital blood (ocular blood) collection was performed every three days from day 7 after tumor cell transplantation, followed by measuring Foxp3-expressing dendritic cells (expressed as fxDC or Foxp3⁺ DC) in the blood (see Reference Examples 4 and 5). The results are depicted in FIG. 1. FIG. 1 shows results of monitoring fxDC populations in the blood of Foxp3^{GFP} mice during tumor growth, wherein fxDC was estimated at each time point for blood collected from the ocular veins of the mice in each group (n=30 for three groups, 10 mice per group). As shown in FIG. 1, the percentage of fxDC in the blood of the tumor mouse models appears to increase with tumor growth.

In addition, measurement was made of fxDC in blood DC (b-DC) (see Reference Examples 4 and 5) from healthy donors (HD) and cancer patients (glioblastoma (GBM, stages 3 and 4), colorectal cancer (CC, stage 2(CC2), 3(CC3) and 4(CC4)) and gastric cancer (GC, stage 2(GC2), 3(GC3) and 4(GC4)) (see Reference Example 2). The results are depicted in FIG. 2a. As shown in FIG. 2a, fxDC distributions in the blood of human cancer patients were increased in proportion to cancer progression, like the mouse tumor models.

In addition, measurement was made of fxDC distributions in the blood of 5-7 tumor mice to which various tumors (EL4; lymphoma, LLC; Lewis lung carcinoma, 266-6; pancreatic cancer, CT-26; colorectal carcinoma, 4T-1; breast cancer) had been transplanted as described above, and the result is depicted in FIG. 2b. As shown in FIG. 2b, fxDC was abundantly found in tumor mouse blood.

### EXAMPLE 2

### Assay for Tumor Growth Inhibition by fxDC Inhibition

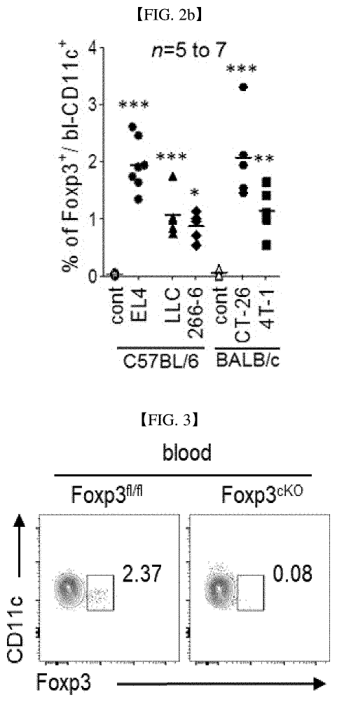

To investigate the effect of fxDC on tumor growth, first, DC-specific Foxp3-knockout mice (CD11c-Cre×Foxp3^{fl/fl}: hereinafter referred to Foxp3^{cKO}) were constructed (see Reference Example 1), followed by injecting tumor cells thereto to prepare tumor mice before measurement of blood fxDC (see Reference Examples 4 and 5). The results are depicted in FIG. 3. As can be seen in FIG. 3, fxDC was depleted from the blood of Foxp3^{cKO} mice.

From seven days after injection of EL4 lymphoma tumor cells (5×10⁵ cells) thereto, wild-type mice (Foxp3^{fl/fl}; TB mouse in which Foxp3 had not been knocked out) and Foxp3^{cKO} mice were monitored every three days for tumor growth. The results are depicted in FIG. 4. FIG. 4 shows plots of tumor volumes vs. times (left) and tumor weights on day 23 after tumor transplantation (right). As shown in FIG. 4, wild-type mice (Foxp3^{fl/fl}) gradually increased in tumor size whereas tumors in the fxDC-depleted Foxp3^{cKO} mice (As for fxDC depletion, reference is made to the result of FIG. 3) grew slightly until day 17, but were completely removed after day 30. These results indicate that the knockout of Foxp3 in a dendritic cell-specific manner or the depletion of fxDC leads to a therapeutic effect on tumors.

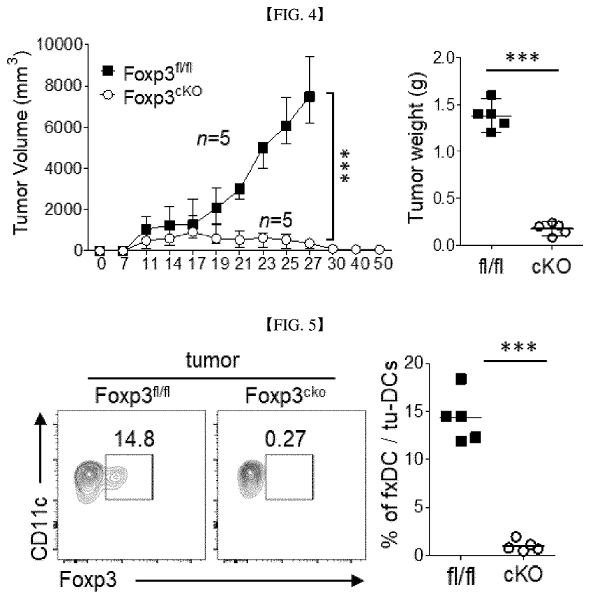

On day 17 after tumor transplantation, fxDC distributions in tumor tissues of Foxp3^{cKO} mice, which were found to stop tumor growth in FIG. 4, were measured (see Reference Examples 4 and 5) and the results are depicted in FIG. 5. As shown in FIG. 5, most fxDC disappeared from Foxp3^{cKO} mice in which tumor growth had actually been repressed.

In addition, the same experiment as for EL4 lymphoma was applied to various solid cancers (266-6: pancreatic cancer, LLC: Lewis lung carcinoma, EG7: OVA expressing EL4 lymphoma) to measure tumor volumes. The measurements are depicted in FIG. 6. As shown in FIG. 6, tumor suppression effects on various solid cancers were found to be remarkably better in fxDC-depleted mice than wild-type mice, as on EL4 lymphoma.

### EXAMPLE 3

### Assay for Increase of CD8⁺ T Cells and Cytotoxicity Against Tumor Cells by fxDC Inhibition

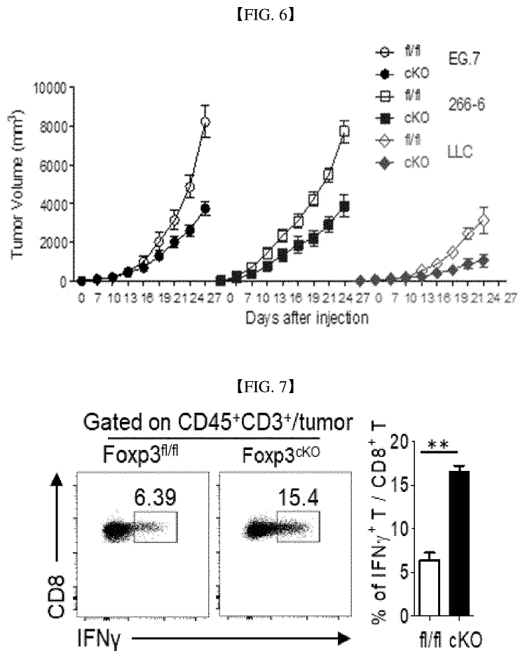

CD8⁺ T (Tc1) cells play a crucial role in anti-cancer immunity and directly induce the apoptosis of tumor cells (cytotoxic CD8⁺T-cell). CD8⁺ T cells in the tumor of fxDC-depleted Foxp3^{cKO} mice amounted to about 35.6%, which was observed to be a great increase over the proportion (about 16.3%) of CD8⁺ T cells in wild-type mice (Foxp3^{fl/fl}). Among CD8⁺ T cells in tumor tissues of fxDC-depleted Foxp3^{cKO} mice, proportions of IFN-gamma-expressing CD8⁺ T cells (IFN-gamma⁺ CD8⁺ T cells; cytotoxic CD8⁺ T-cells) were measured, and the results are depicted in FIG. 7. As shown in FIG. 7, the proportion of the cytotoxic CD8⁺ T-cells in fxDC-depleted Foxp3^{cKO} mice was 2.5 times as large as that in wild-type mice (Foxp3^{fl/fl}). The results suggest the regulatory effect of fxDC on cytotoxic CD8⁺ T-cells (upregulation of cytotoxic CD8⁺ T-cell by fxDC depletion).

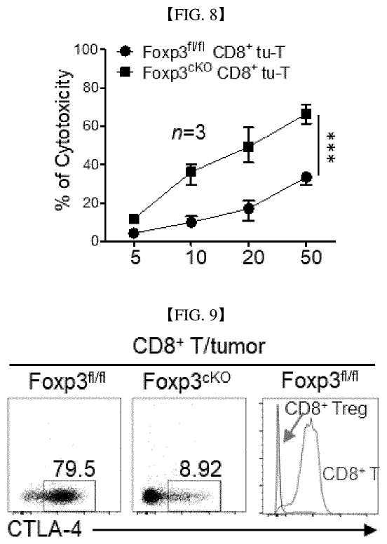

Investigation was made to see whether CD8⁺ T cells in tumor tissues directly induce the inhibition of tumor growth (death of tumor cells). In this regard, CD8⁺ T cells were isolated from tumor tissues of wild-type mice (Foxp3^{fl/fl}) and fxDC-depleted Foxp3^{cKO} mice and co-cultured with tumor cells to measure cytotoxic effects on the tumor cells. Cytotoxicity was measured with reference to the CTL (Cytotoxic T Lymphocytes) activity assay (see Reference Example 7). The results are depicted in FIG. 8. As shown in FIG. 8, CD8⁺ T cells isolated from Foxp3^{cKO} mice exhibited remarkably higher cytotoxicity against tumor cells than those isolated from wild-type mice, indicating that Foxp3 knockout induces the production of CD8⁺ T cells which, in turn, increases death rates of tumor cells, showing a tumor suppressive effect.

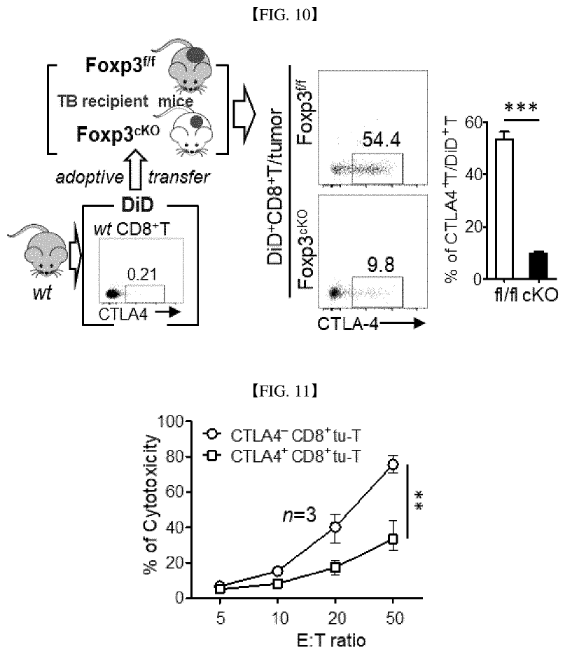

To identify the mechanism through which CD8⁺ T-cells enhance CTL activity (cytotoxicity) in fxDC-depleted mice, investigation was made as to the expression of various cell surface immune activation/suppression molecules. Among them, expression levels of CTLA4 (cytotoxic T-lymphocyte-associated protein 4) in CD8⁺ T cells were compared between tumor tissues of fxDC-depleted TB mice and wild-type mice (TB), and the results are depicted in FIG. 9. As shown in FIG. 9, a great reduction was detected in the expression level (about 8.92%) of CTLA4 in CD8⁺ T cells of tumor tissues of fxDC-depleted TB mice, compared to that in wild-type TB mice (about 79.5%).

The mechanism in which fxDC regulates the CTLA4 expression of CD8⁺ T cells was investigated. For this, CD8⁺ T cells of (Donor T cells: DiD stained) of a normal mouse (not transplanted with tumor) were subjected to adoptive transfer (AT) (see Reference Example 8) to tumor recipient wild-type mice and Foxp3^{cKO} mice via the tail vein. Three days after AT, CTLA4-expressing CD8⁺ T cells (CTLA4⁺ CD8⁺ T cells) were counted among Donor T cells (DiD⁺ CD8⁺ T cells) in the tumor tissue. The results are depicted in FIG. 10. As shown in FIG. 10, a great reduction was detected in the expression level of CTLA4 in Donor CD8⁺ T cells from fxDC-depleted mice.

Examination was made to see whether CTLA4 expression in CD8⁺ T cells regulates the CTL response essential for anticancer immunity (cytotoxicity against tumor cells). In this context, CTLA4⁺ CD8⁺ T cells and CTLA4⁻ CD8⁺ T cells were isolated from EL4 tumor of EL4 TB mice and then assayed for CTL activity, with the tumor cells (EL4) serving as target cells. The results are depicted in FIG. 11. As shown in FIG. 11, CTLA4⁺ CD8⁺ T cells, which express CTLA4, were observed to have remarkably reduced CTL activity, compared to CTLA4⁻ CD8⁺ T cells, which do not express CTLA4.

The results demonstrate that fxDC formed by tumors and tumorous environments induces intratumoral CD8⁺ Treg cells (see Example 5 below) which, in turn, suppress the activity of CTL rushing for tumor clearance and thus are involved in the continuous growth of tumor. When fxDC is depleted, CTLA4 inhibitory of CTL activity decreases in expression level. Thus, tumor-specific CTL activity is not suppressed, but induces effective anticancer immunity, thereby remarkably inhibiting tumor growth. Therefore, the depletion of fxDC in tumor patients is expected to elicit excellent effects of inhibiting cancer growth and/or treating cancer by inducing effective anticancer immunity.

Taken together, the data obtained herein show that CD8⁺ Treg induced by fxDC inhibits T cell growth and CTL activity responsible for anticancer immunity, suggesting that the depletion of fxDC could bring about an improvement in T cell immunity and/or anticancer immunity.

### EXAMPLE 4

### Preparation of CD8⁺ Treg by Co-Culturing fxDC and CD8⁺ T Cells

EL4 tumor cells were s.c. injected at a dose of 5×10⁵ cells into wild-type normal mice. Fourteen days post injection, PBMCs were isolated from blood of the mice. To this end, a 15-ml conical tube (Hyundai micro, Cat. # H20050) was charged with 1 ml of Ficoll-Paque (GE healthcare, Cat. #17-5442-02) which was then overlaid with the same volume of blood or buffy coat with care not to mix them. Density gradient centrifugation was performed for 30 min at 2500 rpm in a multipurpose centrifuge (Gyrozen, Cat. #1580MGR) with acceleration (ACC) and deceleration (DCC) set to be 1 and 0, respectively. After centrifugation, the plasma in the uppermost layer and the mononuclear cells in the middle layer were separated from each other. CD11c⁺ dendritic cells were isolated from the separated mononuclear cells with the aid of CD11c-Microbeads.

After being excised from normal mice, the spleen was mashed through a cell strainer to separate the cell mass into single cells from which all erythrocytes were then removed using an RBC lysis buffer. CD8⁺ T cells were isolated with Microbeads. The CD8⁺ T cells thus obtained were seeded at a density of 2.5×10⁵ cells per well onto CD3/CD28-coated 96-well plates.

The dendritic cells isolated from blood beforehand were aliquoted into the CD8⁺ T cell-containing 96-well plates. After co-culture for three days, the CD8⁺ T cells were harvested and used in the separation and assay of Examples 5 and 6, below.

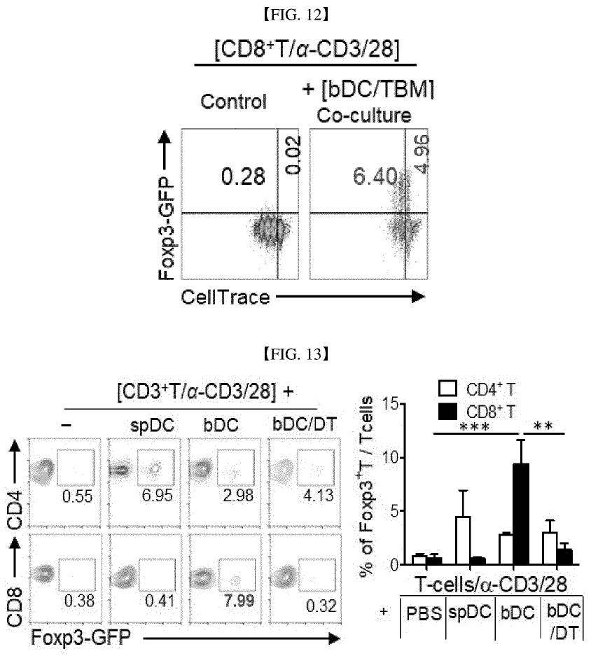

In order to identify the induction of CD8⁺ Treg by co-culturing fxDC and CD8⁺ T cells as described above, CTV-labeled CD8⁺ T cells isolated from Foxp3^{GFP} TB mice and pre-activated with an anti-CD3/28 antibody were co-cultured with DC isolated from the blood of Foxp3^{GFP} TB mice and Foxp3⁺ CD8⁺ Treg distributions were measured. The results are depicted in FIG. 12. As shown, CD8⁺ Treg was induced by co-culturing fxDC and CD8⁺ T cells.

### EXAMPLE 5

### Assay for CD8⁺ Treg Induction by fxDC

The induction of CD8⁺ Treg (CD8⁺ regulatory T cells) by fxDC was investigated. For this, splenic DC (spDC), fxDC-containing blood DC (bDC), and fxDC-depleted blood DC (target cells are depleted by treating Foxp3^{-DTR} mice with diphtheria toxin (DT); fxDC-depleted (DT-treated) bDCs (bDC/DT)) were each co-cultured with T cells pre-activated with an anti-CD3/28 antibody, followed by measuring population ratios of Foxp3⁺ CD4⁺ and CD8⁺ T cells to overall T cells. The measurements are depicted in FIG. 13. As shown in FIG. 13, fxDC-depleted blood DC could not induce CD8⁺ Treg at all.

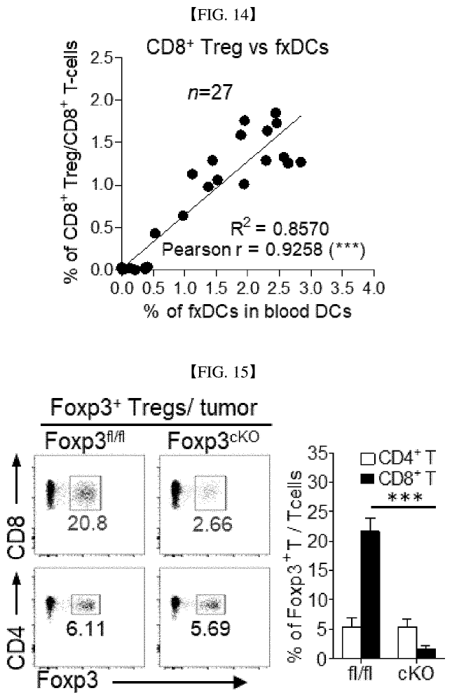

A relationship between fxDC-induced CD8⁺ Treg and tumor growth was assayed. In this regard, 27 Foxp3^{GFP} mice were simultaneously inoculated with EL4 cells to construct TB mice which were then sacrificed one a day for analysis. Measurements of fxDC and CD8⁺ Treg in the blood of the TB mice are plotted for proportions (%) of fxDC in blood DC on the X-axis versus proportions (%) of CD8⁺ Treg in blood (% of CD8⁺ Tregs/CD8⁺ T-cells) on the Y-axis in FIG. 14. As shown in FIG. 14, CD8⁺ Treg was found to increase in proportion to fxDC, which increased with tumor growth in blood of TB mice.

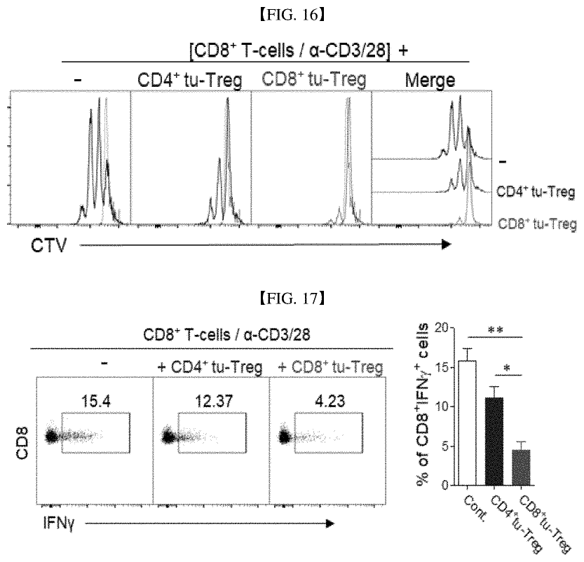

Based on the result that tumors in fxDC-depleted mice had grown, but disappeared after a certain time (see FIG. 4), distributions of Foxp3⁺ CD4⁺ and CD8⁺ Treg in tumor tissues of Foxp3^{cKO} TB mice and wild-type TB mice were measured and the measurements are depicted in FIG. 15. As shown in FIG. 15, CD8⁺ Treg was greatly reduced in tumor tissues of fxDC-depleted mice, compared to wild-type mice, but CD4⁺ Treg cells were independent of the presence or absence of fxDc.

### EXAMPLE 6

### Assay for CD8⁺ Treg Activity of Inhibiting T Cell Growth and Promoting Tumor Growth

Effects of fxDC-induced CD8⁺ Treg on T cell immunity and anticancer immunity were investigated. For this, splenic CD8⁺ T-cells were stimulated with an anti-CD3/28 antibody and then co-cultured with tumor-CD4⁺Treg (tu-CD4⁺ Treg) cells or tumor-CD8⁺ Treg (tu-CD8⁺ Treg) cells, which were both isolated from tumors of Foxp3^{GFP} TB mice, for three days before measurement of CD8⁺ T cell growth and IFN-gamma⁺ cells (see Reference Examples 5 and 6).

FIG. 16 shows measurements for the growth of CD4⁺ Treg (tu-CD4⁺ Treg) cells and CD8⁺ Treg (tu-CD8⁺ Treg) cells, illustrating that CD8⁺ Treg cells repress T cell growth at a higher level than CD4⁺ Treg cells when anti-CD3/28 antibody-treated (pre-activated) T cells are co-cultured with CD8⁺/CD4⁺ Treg cells.

FIG. 17 shows levels of IFN-gamma⁺ T cells after anti-CD3/28 antibody-treated (pre-activated) T cells are co-cultured with CD8⁺/CD4⁺ Treg cells, illustrating that co-culturing of anti-CD3/28 antibody-treated (pre-activated) T cells and CD8⁺/CD4⁺ Treg cells greatly reduces the level of IFN-gamma-expressing CTL (CD8⁺ IFN-gamma⁺ T cells).

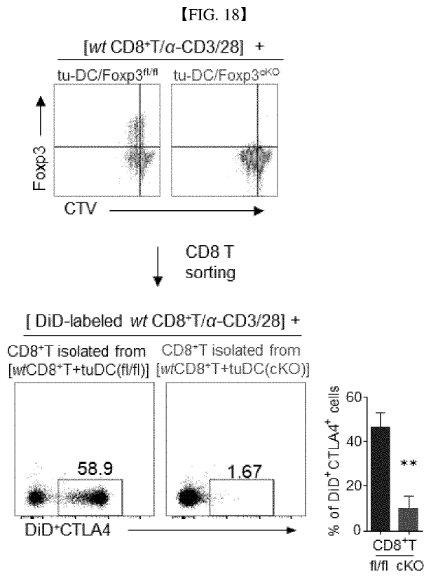

As demonstrated in the previous Examples, CTLA4⁺ CD8⁺ T cells lose CTL activity (see FIG. 10). In this context, examination was made to see whether CD8⁺ Treg directly induces CTLA4⁺ CD8⁺ T cells. For this, CD8⁺ Treg induced in vitro by fxDC was co-cultured with CD8⁺ T cells of normal mice, followed by measuring CTLA4⁺ CD8⁺ T cell levels. Briefly, CD8⁺ T-cells isolated from wild-type (normal) mice were stimulated with an anti-CD3/28 antibody and then co-cultured with to-DC isolated from tumor of Foxp3^{f/f} and Foxp3^{cKO} TB mice for three days before purification of to-DC-induced CD8⁺ T cells. These cells were co-cultured with DiD-labeled wild-type CD8⁺ T-cells for three days, followed by measuring expression levels of CTLA-4 in CD8⁺ T-cells. The results are depicted in FIG. 18. As shown in FIG. 18, CTLA4⁺ CD8⁺ T cell levels in Foxp3^{cKO} TB mice were remarkable reduced compared to those in wild-type TB mice, implying that fxDC-induced CD8⁺ Treg directly induces CTLA4⁺ CD8⁺ T cells.

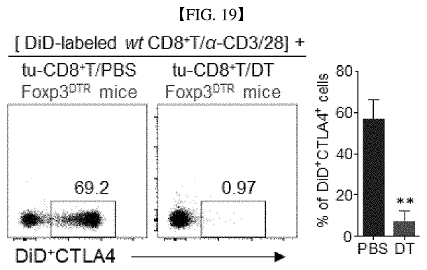

Wild-type CD8⁺ T cells were labeled with DiD, stimulated with an anti-CD3/28 antibody, and co-cultured with DT-treated to-CD8⁺ T-cells (CD8 Treg-depleted) or PBS-treated tu-CD8⁺ T-cells, which were both isolated from tumors of Foxp3^{DTR} TB mice, for three days before measurement of CTLA4⁺ CD8⁺ T cell levels. The measurements are depicted in FIG. 19. As shown in FIG. 19, depletion of CD8⁺ Treg by treatment with DT did not induce CTLA4⁺ CD8⁺ T cells at all.

Taken together, the data obtained above demonstrate that fxDC-induced CD8⁺ Treg inhibits CTL activity by inducing CTLA4 expression in CTL, which directly attacks cancer cells, suggesting that the depletion of CD8⁺ Treg could enhance anticancer immunity and thus implement more effective cancer therapy, together with the usefulness of CD8⁺ Treg as an anticancer therapy target.

1-9. (canceled)

10. A method for treatment of cancer, the method comprising a step of administering an inhibitor against a Foxp3-expressing dendritic cell to a patient in need thereof

11. The method of claim 10, wherein the inhibitor against a Foxp3-expressing dendritic cell is least one selected from the group consisting of antibodies, cytotoxic drugs, antibody-cytotoxic drug conjugates, and antibody-magnetic particles or is in a form of a nano-delivery system comprising the inhibitor.

12. The method of claim 10, wherein the patient is a cancer patient having a Foxp3-expressing dendritic cell detected in a tumor tissue or blood thereof.

13. A method for inhibiting a CD8-positive regulatory T cell, the method comprising a step of administering an inhibitor against a Foxp3-expressing dendritic cell to a patient in need thereof.

14. The method of claim 13, wherein the inhibitor against a Foxp3-expressing dendritic cell is at least one selected from the group consisting of antibodies, cytotoxic drugs, antibody-cytotoxic drug conjugates, and antibody-magnetic particles or is in a form of a nano-delivery system comprising the inhibitor.

15. The method of claim 13, wherein the patient is a cancer patient having a Foxp3-expressing dendritic cell detected in a tumor tissue or blood thereof.

16. A method for treatment of cancer, the method comprising a step of administering an inhibitor against a CD8-positive regulatory T cell to a patient in need thereof.

17. The method of claim 16, wherein the inhibitor against a CD8-positive regulatory T cell is at least one selected from the group consisting of antibodies, cytotoxic drugs, antibody-cytotoxic drug conjugates, and antibody-magnetic particles or is in a form of a nano-delivery system comprising the inhibitor.

18. The method of claim 16, wherein the patient is a cancer patient having a CD8-positive regulatory T cell detected in a tumor tissue or blood thereof.

19. A method for screening an anticancer agent, the method comprising the steps of:
contacting a Foxp3-expressing dendritic cell, a CD8-positive regulatory T cell, or both with a candidate compound;
measuring levels of a Foxp3-expressing dendritic cell, a CD8-positive regulatory T cell, or both; and
defining the candidate compound as a candidate for an anticancer agent in a case where the levels of a Foxp3-expressing dendritic cell, a CD8-positive regulatory T cell, or both decrease, compared to those measured before the contact with the candidate compound.

20. A method for preparation of a CD8-positive regulatory T cell, the method comprising a step of co-culturing a Foxp3-expressing dendritic cell and a CD8-positive T cell.

21. A method for providing information for cancer diagnosis or cancer prognosis identification, the method comprising a step of detecting a Foxp3-expressing dendritic cell, a CD8-positive regulatory T cell, or both in a biological sample isolated from a patient.

22. A method for providing information on the monitoring of anticancer therapy efficacy, the method comprising a step of detecting a Foxp3-expressing dendritic cell in a biological sample isolated from a patient.
